(19) United States
(12) Patent Application Publication
(10) Pub. No.: US 2019/0024076 A1
(43) Pub. Date: Jan. 24, 2019

(54) Semi-dry bead beating method for microbial lysis and device for performing same

(71) Applicant: Shoreline Biome, LLC, Farmington, CT (US)

(72) Inventors: Mark Driscoll, Wallingford, CT (US); Thomas Jarvie, Branford, CT (US); Ryan Beach, Simsbury, CT (US); Jeff Whynall, Killingworth, CT (US)

(21) Appl. No.: 16/036,006

(22) Filed: Jul. 16, 2018

Related U.S. Application Data
(60) Provisional application No. 62/533,821, filed on Jul. 18, 2017.

(51) Int. Cl. C12N 15/10; C12N 1/06; C12M 1/02

(52) U.S. Cl. CPC C12N 15/1003; C12M 1/264; C12M 1/02; C12N 1/066

(57) ABSTRACT

Disclosed are methods and devices for lysing cells to release extract genomic DNA (gDNA). The methods use a mixture of microscopic glass beads and cells (for example, spores) that form a semi-dry cake that clings to a larger metal ball and the sides of the tube during bead beating lysis, greatly improving the efficiency of the bead beating process. The devices produce a chaotic motion which ensures that sufficient force is generated to open the cells, and that the metal ball impacts are distributed across the interior surface of the container so that all of the cell mixture is subjected to sufficient impacts to break the cells. As a result, spores and other difficult-to-lyse microbes, can be opened in seconds. The method reduces the number of steps and hands-on time by rapidly opening difficult to lyse cells, while preserving the integrity of the DNA.

### CROSS-REFERENCE TO RELATED APPLICATIONS

This application claims priority to patent application No. 62/533,821, filed Jul. 18, 2017, the content of which is incorporated herein by reference in its entirety.

### FIELD OF THE DISCLOSURE

Disclosed are a method and device for lysing cells to release or extract genomic DNA (gDNA) from inside cells. The disclosed method uses a mixture of microscopic glass beads and cells (for example, spores) that form a semi-dry cake that clings to a larger metal ball and the sides of the tube during bead beating lysis, greatly improving the efficiency of the bead beating process. Since the cells are caked onto the glass beads and metal ball, each time the ball strikes the side of the container, cells are impacted. The device is designed to produce a chaotic motion with a dual purpose, first ensuring that sufficient force is generated to open the cells, and second that the metal ball impacts are distributed across the interior surface of the container so that all of the cell mixture is subjected to sufficient impacts to break the cells. The result is that spores and other microbes that typically need to be boiled or beaten for up to 30 minutes to achieve adequate lysis, can be opened in seconds using the disclosed semi-dry bead beating method. The method reduces the number of steps and hands-on time by rapidly opening difficult to lyse cells, while preserving the integrity of the DNA.

### BACKGROUND OF THE DISCLOSURE

Many cell-based and DNA-based analytical methods require releasing cell contents including DNA from inside a cell to facilitate analysis. Opening the cells to release the contents is called ‘lysis.’ For example, methods used to investigate the microbiome using DNA sequencing techniques first require lysis of microbes so the DNA can be extracted. Most microbiomes are communities of bacteria, archaea and fungi that vary tremendously in their susceptibility to lysis techniques. Differential susceptibility presents a significant problem to microbial population researchers, who want to ensure that the toughest (usually Gram-positive) and the easiest (usually Gram-negative) to lyse bacteria are represented in proportion to their population in the original sample. Unfortunately, most microbial lysis protocols work well for some microbes, but poorly for others. (Comparison of lysis techniques for microbiome—Sanqing Yuan, Dora B. Cohen, Jacques Ravel, Zaid Abdo, Larry J. Forney. Evaluation of Methods for the Extraction and Purification of DNA from the Human Microbiome. PLoS ONE 7(3): e33865. doi:10.1371/journal.pone.0033865; Additionally, rapid and simple alkaline lysis techniques used to recover plasmid DNA typically also remove the microbial genomic DNA, which is the target for microbiome screening (Alkaline Lysis opens cells but removes gDNA—Birnboim, H C. and Doly, J., A rapid alkaline extraction procedure for screening recombinant plasmid DNA, Nucleic Acids Res. 7(6), 1979, 1513-1524; in contrast, KOH lysis can be used to recover bacterial genomic DNA Raghunathan, Arumugham et al. “Genomic DNA Amplification from a Single Bacterium.” Applied and Environmental Microbiology 71.6 (2005): 3342-3347. PMC. Web. 29 September 2016). There are multiple lysis techniques known in the art that attack cellular integrity based on different biochemical methods, including lysozyme (enzymatic attack on the peptidoglycan cell wall), strong base (chemical attack), detergent (solubilizes cell membranes), bead beating or shaking (mechanical disruption), and heat DNA extraction methods affect microbiome profiling results: Wagner Mackenzie B, Waite D W, Taylor M W Evaluating variation in human gut microbiota profiles due to DNA extraction method and inter-subject differences. Frontiers in Microbiology. 2015; 6:130. doi:10.3389/fmicb.2015.00130). Most published or commercially available DNA preparation methods use one or more of these methods to lyse cells, usually in sequential steps that can take a significant amount of time, especially when handling many samples at once. While individual lysis methods are usually sufficient for applications where incomplete or partial lysis yields sufficient DNA for the protocol being performed, they often do not yield DNA from microbiome samples in proportion to the original community, and may fail to lyse certain microbes altogether. For example, a detergent-based lysis may disrupt a subset of cells with weak cell walls and strong cell membranes, but not open detergent-resistant microbes with strong cell walls, leading to under-representation or absence of DNA from detergent resistant cells in the resulting DNA preparation. In another example, bead beating of microbes sufficient to lyse cells with strong cell membranes may shear or destroy DNA released early in the process from easily lysed cells. Additionally, the various methods of lysis tend to be incompatible with each other, and need to be performed sequentially if used in combination. For example, lysozyme will not work in the presence of detergents or strong base. Certain detergents precipitate in the presence of strong base. Bead beating is difficult to combine with a heating process. While individual shortcomings may be overcome by running separate lysis protocols in series, this increases the complexity, time, and cost involved. Importantly, detergents such as sodium dodecyl sulfate (SDS) must be removed after lysis, because SDS interferes with downstream DNA manipulation. Additionally, certain microbes may be resistant to lysis protocols run sequentially, depending on protocol order. For example, certain microbes with tough peptidoglycan cell walls may have an outer envelope of lipid bi-layer that protects from an initial treatment with strong base or lysozyme. A simultaneous combination of multiple methods may be effective, or a long sequence of multiple steps, to yield DNA from all microbes in a sample, but the use of a single method that opens all cells would be a significant improvement.

Devices and shakers for conducting bead beating are known in the art. For examples, see Table 1. Bead beaters and shakers capable of processing large numbers of samples tend to be large and heavy with powerful motors and fast moving parts that can be dangerous to users. The devices disclosed herein are small, efficient and safe. Smaller units known in the art typically process a few samples at a time. The devices disclosed herein can process 1-48 samples at once. The devices known in the art use regular or simple motions to process samples, whereas the disclosed devices use a random motion that results in more forceful impacts and wide range of motion that results in more uniform lysis in less time at slower (less dangerous) speeds.

| | # | |
|---|---|---|
| Name | samples | Provider |
| BEADBUG | 3 | Benchmark Scientific |
| | | (Sayreville, NJ) |
| BULLETBLENDERSTORM | 24 | NextAdvance |
| | | (Averill Park, NY) |
| ULTRATURRAX | 1 | IKA |
| BEADBEATER | 1 | BioSpec Products, Inc. |
| | | (Bartlesville, OK) |
| MINILYS | 3 | Bertin Corp (Rockville, MD) |
| BEADBLASTER24 | 24 | Benchmark Scientific |
| | | (Sayreville, NJ) |
| PRECELLYS24 | 24 | Bertin Corp (Rockville, MD) |
| U-MINIBEADBEATER | 1 | BioSpec Products, Inc. |
| | | (Bartlesville, OK) |
| TALBOYS HIGH THROUGH- | 96 | Troemner (Thorofare, NJ) |
| PUT HOMOGENIZER | | |
| OMNI BEAD RUPTOR 12 | 12 | Omni International |
| | | (Kennesaw, GA) |
| GENIE SI-D238 DISRUPTOR | 12 | Zoro Tools |
| | | (Buffalo Grove, IL) |
| BIOSPEC BEAD BEATER | 1 | BioSpec Products, Inc. |
| MILL 909 | | (Bartlesville, OK) |
| MINI BEADBEATER 8 | 8 | BioSpec Products, Inc. |
| | | (Bartlesville, OK) |
| MINI BEADBEATER 1 | 1 | SupplyMyLab |
| FASTPREP-24 5G | 24 | MP Bio (Santa Ana, CA) |
| TISSUELYSER II | 48/192 | Qiagen (Germantown, MD) |
| TISSUELYSER LT | 12 | Qiagen (Germantown, MD) |
| BENCHMARK SCIENTIFIC | 3 | Benchmark Scientific |
| HOMOGENIZER | | (Sayreville, NJ) |
| SPEEDMILL PLUS | 20 | Analytik Jena (Upland, CA) |
| GENO/GRINDER 2010 | 1 | Ops Diagnostics |
| | | (Lebanon, NJ) |
| HT HOMOGENIZER | 96 | Ops Diagnostics |
| | | (Lebanon, NJ) |
| HT 24 | 24 | Ops Diagnostics |
| | | (Lebanon, NJ) |

The methods and devices disclosed herein utilize a bead beating procedure that works rapidly to lyse cells, including even the most difficult microbial spores (Bacillus subtilis spores are described herein as an example), yet preserves DNA of sufficient size to generate the large amplicons needed for applications and techniques where proportional lysis is desired or necessary, such as high resolution microbiome characterization. The result is a simple, rapid protocol that uniformly opens all cells in a sample, yielding a more representative DNA profile across a sample containing different cellular constituents, such as the microbiome.

### BRIEF SUMMARY

Disclosed are methods and devices for lysis of cells, such as bacteria present in microbiomes, that can be completed in a short period of time, e.g., less than a minute, and importantly, yield improved quality and quantities of genomic DNA (gDNA) from difficult to lyse samples, such as bacterial spores.

### BRIEF DESCRIPTION OF THE DRAWINGS

FIG. 1 shows a 2 milliliter microcentrifuge tube containing a single 4.5 millimeter steel ball and 0.05 grams of 100 micrometer glass beads.

FIG. 2 shows the semi-dry cake formed on the outside of the steel ball.

FIG. 3 is a graph showing the release of dipicolinic acid (DPA) following bead beating. DPA is a compound present inside the core of many bacterial spores, is released only after lysis of the spores, and can be detected by fluorescence when combined with TbCl₃. Samples were subjected to bead beating for increasing amounts of time. Each sample consisted of 4 ul of a 1 OD/ml solution of B. subtilis spores in TE. Spores were added to 5 mg of glass beads (Sigma, G4649) in a 2 ml centrifuge tube as shown in FIG. 1. The tubes containing the spore mixtures were subjected to bead beating on the instrument for the indicated amount of time. When bead beating was complete, 150 ul TE was added to the glass bead, from which 100 microliters of the lysate was recovered.75 microliters of lysate from each time point was used in a DPA assay. Lysate was added to a black, flat bottomed 96-well plate containing 50 ul of a solution containing 0.2 uM TbCl3 and 0.1M K-HEPES. The 96-well plate was read in a fluorescent plate reader at 270 nM excitation and 545 nM emission. Relative fluorescence units corresponding to dipicolinic acid fluorescence were recorded for each sample and plotted. Control lysis contained 4 ul of spores boiled for 30 minutes.

FIG. 4 is a graph showing the assessment of the quality and relative quantities of the DNA released from bacterial spores after bead beating. DNA quality was assessed by PCR of the 1500 base V1-V9 region of the bacterial 16S rRNA gene using the Shoreline Biome 16S V1-V9 DNA Purification and PCR Amplification Kit (Shoreline Biome cat#SBV19-16). Relative quantity was assessed by comparing the Cq for each bead beating time point. 5 microliters of the same lysates described in FIG. 3 were mixed with 0.1 ul 1:20 EvaGreen (Biotum, cat#31000), added to PCR reactions and amplified according to manufacturer's instructions using a BioRad CFX 96 Touch thermocycler set to detect Sybr dye. Cq values were recorded for each time point and plotted.

FIG. 5 is a graph showing the release of DPA and the corresponding release of DNA from the spores after lysis by the methods described herein. Spore lysis was measured using the terbium fluorescence assay as in FIG. 3. DNA quantity was measured by qPCR quantification as in FIG. 4. Spores were subjected to bead beating for up to 10 minutes as detailed on the X-axis.

FIG. 6 is a graph showing spore lysis and DNA quantity during boiling lysis of spores. Spore lysis was measured using the terbium fluorescence assay as in FIG. 3. DNA quantity was measured by qPCR quantification as in FIG. 4. Spores were suspended in TE and subjected to 100° C. temperatures (boiling) for the times indicated on the X-axis.

FIG. 7 is a graph showing the release of DPA was measured for each trial as an indicator of spore lysis when time and intensity of shaking was varied.

FIG. 8a is a diagram of the bead beater prototype. Elements of the prototype discussed below in the ‘Detailed Description’ section are labeled.

FIG. 8b is a diagram of the details of the striker bar and slot region of the ‘free element’ as discussed below in the ‘Detailed Description’ section.

### DETAILED DESCRIPTION

The subject matter that is regarded as the invention is particularly pointed out and distinctly claimed in the claims at the conclusion of the specification. The foregoing and other objects, features, and advantages of the invention will be apparent from the following detailed description taken in conjunction with the accompanying drawings.

DNA-based analytical methods, for example, the methods described in U.S. patent application Ser. No. 15/372,588 titled “Methods for DNA Preparation for Multiplex High Throughput Targeted Screening” by Mark Driscoll and Thomas Jarvie, that is incorporated herein by reference in its entirety, generate DNA by lysing the cells in the target microbiome, after which the resulting DNA is used as template in PCR amplification targeting the 16S, 23S or other rRNA (ribosomal RNA) gene sequences, present in all bacteria and archaea. Microbes can be identified using their rRNA gene sequence, which varies slightly in most, if not all, bacteria and archaea. The variation in rRNA gene sequence means that individual species of bacteria and archaea have characteristic DNA variations in the rRNA gene that serve as identifiers, or fingerprints, for that species. Kits, protocols and software enable comprehensive fingerprinting of the microbes in a sample, and permit simultaneous rRNA fingerprinting of many samples at once, at high resolution, using rRNA gene sequences. Known microbes can be identified after sequencing by mapping the rRNA gene DNA sequences to a microbial genomic database. Unknown microbes will contain rRNA sequences that are different from any of the microbes in the database, but can be tracked using their unique rRNA sequence. In addition, the number of reads obtained for each microbe in a sample can reveal the relative abundances of each microbe in a sample. The relative abundance can be an important indicator of the state of each individual microbiome. Lysis techniques that change relative abundances of microbes, or leave out certain microbes altogether, can lead to sequencing results that incorrectly characterize the state of the microbiomes being studied. The invention as described is an improved lysis method for achieving the correct relative abundances of microbes from a sample.

As used herein, “non-periodic motion” means any motion that does not have a regular period of repetition or orbital motion and repeats itself at non regular intervals of time.

As used herein, “random motion” and “chaotic motion” are used interchangeably to mean any motion in which the speed and three-dimensional direction continually change.

As used herein “semi-dry cake” means a sample of biologic cells having 1% to 30% weight of sample (for example, spores or microbes) per weight of moisture.

As used herein, “micron” and “micrometer” are used interchangeably to mean a unit of length equal to one millionth of a meter.

Disclosed herein is a device for shaking samples in a non-periodic motion and random motion simultaneously, having a stroke length of from about 1 cm to about 2.5 cm and a speed of from about 5 Hz to about 50 Hz. In some embodiments, the device is configured to hold samples in microfuge tubes or microtiter plates or other vessels suitable for high-throughput analysis. In some embodiments, the number of tubes that can be processed simultaneously is between one and 96. In some embodiments, the microtiter plate is selected from the group consisting of: 8, 16, 24, 46, 96, or 384 well plate. In some embodiments the volume of the tubes or wells in the microtiter plates is between 200 microliters to 2 ml.

Disclosed herein is a method for lysing biologic cells in a sample to release DNA from the cells, comprising the sequential steps of: (a) mixing a first aqueous solution or semi-solid sample containing one or more biologic cells with (i) a 4.5 mm steel (or other biocompatible material) ball; and (ii) ˜100 micron beads (glass or other material 4 or greater on the Mohs hardness scale) to form a semi-dry cake and (b) shaking the mixture of (a) in a non-periodic and random motion for a period of time effective to release DNA from the cells; (c) resuspending the semi-dry cake in a second aqueous solution and allowing the beads to settle out; (d) recovering the second aqueous solution from the settled solid components in the mixture, wherein DNA released from the biologic cells is present in the recovered second aqueous solution.

In some embodiments, the first aqueous solution is water. In some embodiments, the first aqueous solution contains Tris or other buffers, salts or detergents to control pH or limit biological activity. In some embodiments, the first solution is a non-aqueous liquid sufficient to form a semi-dry cake when added to the beads. In some embodiments, the first aqueous solution contains one or more biologic cells.

In some embodiments, the steel ball has a diameter of from about 2 millimeters to about 10 millimeters. In some embodiments, the steel ball has a diameter of about 4.5 millimeters. In some embodiments, the glass beads have a diameter of from about 10 micrometers to about 300 micrometers. In some embodiments, the glass beads have a diameter of about 100 micrometers. In some embodiments, the glass beads are added to the first aqueous solution at a concentration of about 83% glass beads by weight (for example, 10 ul (10 mg) sample to 50 mg beads).

In some embodiments, the glass beads are added to the aqueous solution at a concentration of from 40% to 99% by weight.

In some embodiments, the shaking is conducted for a period of from about 5 seconds to about 10 minutes.

In some embodiments, the shaking is conducted for 2 minutes.

In some embodiments, cell lysis is measured by terbium fluorescence of dipicolinic acid, which is co-located with DNA inside spores

In some embodiments, the release of DNA during lysis is followed by UV spectroscopy (OD₂₆₀).

In some embodiments, the release of DNA during lysis is determined by DNA fluorescence assays.

In some embodiments, the separation of steel and glass beads from the second aqueous solution is conducted by a method selected from the group consisting of: centrifugation, filtration and gravity settling.

In some embodiments, the biologic cells originate from a sample selected from the group consisting of: feces, cell lysate, tissue, blood, tumor, tongue, tooth, buccal swab, phlegm, mucous, wound swab, skin swab, vaginal swab, or any other biological material or biological fluid originally obtained from a human, animal, plant, or environmental sample, including raw samples, complex samples, mixtures, and microbiome samples.

In some embodiments, the biologic cells originate from an organism selected from the group consisting of: spores, biofilms, multicellular organisms, unicellular organisms, prokaryotes, eukaryotes, microbes, bacteria, archaea, protozoa, algae and fungi.

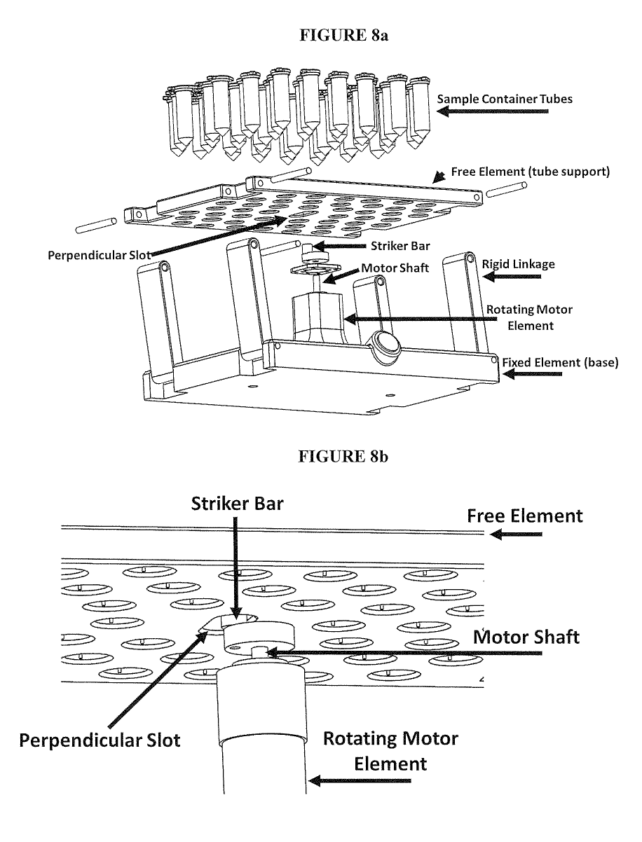

In some embodiments, the shaker unit has a rigid, flat tube or plate support element (free element) into which multiple plates or tubes can be loaded (FIGS. 8a and 8b). In some embodiments, up to 48 sample container tubes can be loaded into the free element. In the preferred embodiment, the free element is connected to a fixed element (base) by rigid linkages that generally constrain motion of the free element to two degrees of freedom and with a slot arranged perpendicular to each degree of freedom. A rotating motor element is mounted to the base element and transfers energy to the free element by means of a striker bar periodically making contact with the slot in the free element. This striker bar is mounted to the motor shaft at one end, and free on the opposing end. In the preferred embodiment, the width of the slot in the free element is substantially larger than the free end of the striker bar, shaking samples in a non-periodic motion and random motion simultaneously. The discontinuous and non-linear chaotic relationship of motion between the free element and the rotating striker bar results in a generally elastic collision between the striker bar and the free element slot that provides for a periodic rapid acceleration of the free element in both degrees of freedom, increasing the force of the strike, and distributing both the cell cake and the steel ball impacts at random across the inside of the sample container. The overall random action enables complete cell lysis in seconds.

### EXAMPLE

The following is an illustrative embodiment of the cell lysis methods disclosed herein:

Step 1: 2 microliters of B. subtilis spores (1 OD/ml) were suspended in 8 ul water to form a 10 μl mixture.

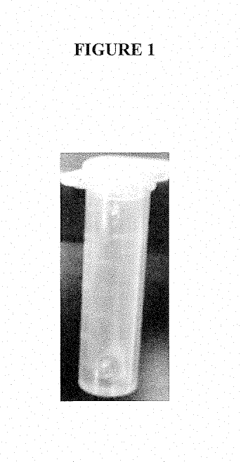

Step 2: A 2 ml microcentrifuge tube (Dot Scientific #RN2000-GMT) was prepared, containing a single 4.5 mm steel ball and 0.05 g glass beads (acid washed, 100 um, Sigma G4649) (see FIG. 1).

Step 3: 10 μl of spore suspension was added to steel bead/glass bead mixture.

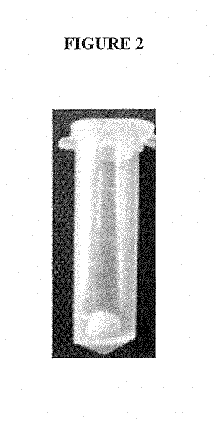

Step 4: Tube was placed in shaker. Semi-dry cake forms on outside of bead (see FIG. 2) during shaking so that each hit of the bead against the tube wall is impacting the glass bead cake containing the spores and cells. Cake also forms on the side wall of the tube during beating.

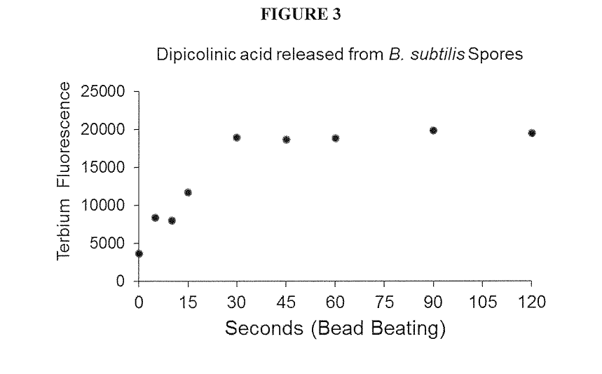

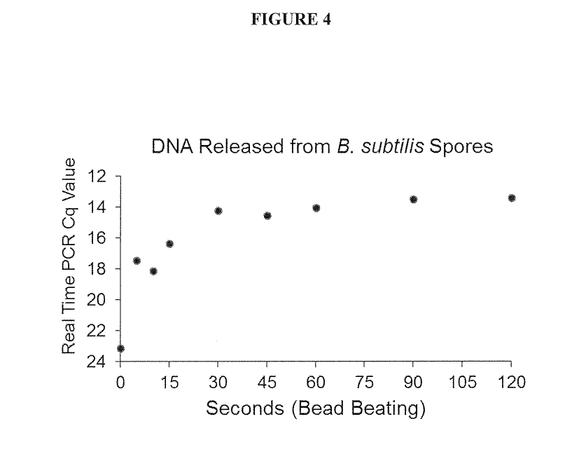

Step 5: The lysis of spores releases dipicolinic acid (DPA), which can be measured via terbium fluorescence. FIG. 3 shows the release of DPA and FIG. 4 shows the release of DNA following bead beating. Control lysis contained spores boiled for 30 minutes. Only DPA release is shown for Control Lysis, because boiling for 30 minutes opens the spores but also selectively destroys the DNA.

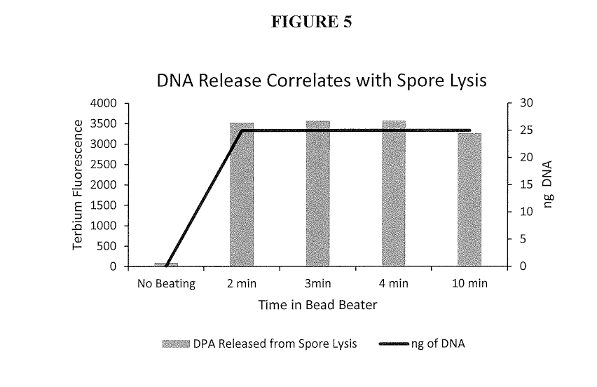

Step 6: DNA quality was assessed by PCR of the 16S rRNA gene (see FIG. 5), a 1500 base amplicon that is frequently used to identify microbes. This amplicon was chosen as an indicator of DNA quality because of it is longer than many typical amplicon targets, so if it is present intact, it shows that the DNA is of sufficient quality for most PCR or sequencing applications. Bead beating in the shaker was extended beyond what was needed to lyse spores in order to test the rate of accumulation of DNA damage. Spore lysis was followed using the terbium assay for DPA as in FIG. 3, which reached a maximum by 2 minutes. Real time PCR was performed on the samples as described in FIG. 4. The real time PCR produced indistinguishable Cq values of 17 cycles for 2, 3, 4 and 10 minutes of bead beating, in contrast to the Cq of 25 for no bead beating. This 8 cycle difference indicates that there was a 250× increase in DNA concentration from the 0.1 ng total included with the control (no bead beating) reaction. Furthermore, it should be noted that there was no change to the Cq after 10 minutes of bead beating, showing that the additional 8 minutes of bead beating not affect the quality of the amplifiable template DNA released from the spores.

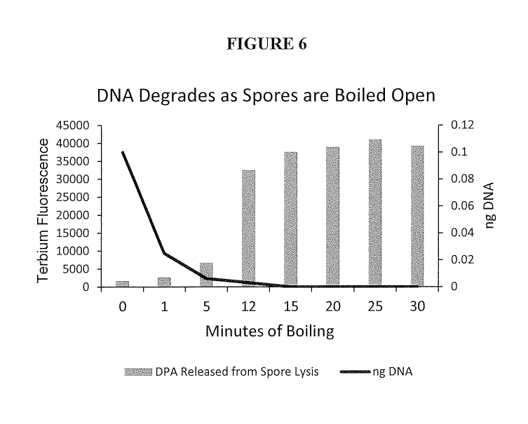

As a comparison, spore lysis and DNA quantity were followed during boiling lysis of spores as shown in FIG. 6. In contrast to the rapid increase in DNA quantity seen with semi-dry bead beating, a rapid decrease in DNA was seen with only 5 minutes of boiling. Additionally, it took 12 minutes for maximal spore lysis by boiling, in contrast to 30-45 seconds of semi-dry bead beating, demonstrating that bead beating is over 20 times faster than boiling lysis. In contrast to bead beating, there was no increase in DNA seen after the DPA fluorescent assay showed that the spores were lysed, showing that the time and temperature required to destroy the spore simultaneously destroys the DNA.

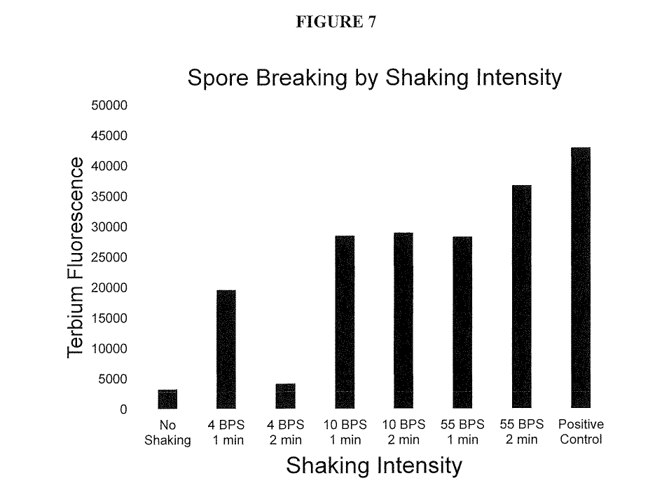

Bead beating parameters were also explored using the novel shaker device disclosed herein. As shown in FIG. 7, the time and speed of shaking was varied, in duplicate, and the release of DPA was measured for each trial as an indicator of spore lysis. Shaking at 10 and 55 beats per second (BPS) showed significant spore lysis. Shaking at approximately 6 BPS was variable with significant lysis for one of the two trials, demonstrating that speeds above 6 BPS are preferred. Spores were boiled for 30 minutes as a positive control.

Shaker intensity was tested up to 55 beats per second with similar results as shown in FIG. 7. The use of the semi-dry cake beating process combined with random shaking action enables a novel shaker design with a small stroke length, decreasing the cost and potential kinetic energy hazard of shakers used for other bead beating processes. This in turn reduces the need for higher powered motors, extensive safety features, and more complex and expensive design, reducing costs and complexity of shaker design, increasing portability and number of samples that can be processed simultaneously while decreasing weight and footprint.

The methods and devices disclosed here may be useful for any applications that require lysis of cells and spores, such as microbiome sequencing (for example, 16S rRNA or other targeted gene profiling), DNA applications (for example, whole genome shotgun profiling), non-DNA applications (for example, protein, RNA, metabolites, and organelles), and diagnostic applications for difficult to lyse pathogens.

One or more embodiments of the present invention have been described. Nevertheless, it will be understood that various modifications may be made without departing from the spirit and scope of the invention. Accordingly, other embodiments are within the scope of the following claims.

What is claimed is:

1. A device for shaking samples in a non-periodic motion and chaotic motion simultaneously, having a stroke length of from about 1 cm to about 2.5 cm, a stroke height from about 0.2 cm to about 1 cm, and a speed of from about 5 Hz to about 55 Hz.

2. The device of claim 1, wherein the device is configured to hold samples in one or more microtiter plates, individual microcentrifuge tubes, or other vessels suitable for low throughput or high-throughput analysis.

3. The device of claim 2, wherein the microtiter plate is selected from the group consisting of: 8, 16, 24, 46, 96, 384 or 1536 well plate.

4. The device of claim 2, wherein the number of microcentrifuge tubes that can be processed simultaneously ranges from 1 to 96.

5. A method for lysing biologic cells in a sample to release DNA from the cells, comprising the sequential steps of:
(a) mixing a first aqueous or non-aqueous solution containing biologic cells with (i) a ball having a diameter of about 2 millimeters to about 10 millimeters, and (ii) beads having a diameter of about 20 micrometers to about 150 micrometers, so as to form a semi-dry cake;
(b) shaking the mixture of (a) in a non-periodic and chaotic motion for a period of time effective to release DNA from the cells;
(c) washing the semi-dry cake with a second aqueous solution to dissolve and/or suspend the cell contents, including the DNA;
(d) separating the second aqueous solution from solid components in the mixture by allowing the beads to settle and recovering the supernatant that contains the DNA and other cellular constituents released in step (b).

6. The method of claim 4, wherein the first solution is (i) an aqueous solution selected from the group consisting of water, a biologic buffer, or another aqueous, or (ii) a non-aqueous solution sufficient to form the semi-dry cake.

7. The method of claim 5, wherein the volume of the biologic cells in the first aqueous solution is about 20% by weight of the combined weight of the beads and the first aqueous solution.

8. The method of claim 5, wherein the ball has a diameter of about 4.5 millimeters.

9. The method of claim 5, wherein the ball is made of a material that is 4 or greater on the Mohs hardness scale.

10. The method of claim 9, wherein the material is steel.

11. The method of claim 5, wherein the beads have a diameter of from about 20 micrometers to about 150 micrometers.

12. The method of claim 11, wherein the beads have a diameter of about 100 micrometers.

13. The method of claim 5, wherein the beads are made of a material that is 4 or greater on the Mohs hardness scale.

14. The method of claim 13, wherein the material is glass.

15. The method of claim 5, wherein the beads are added to the first aqueous solution at a concentration of about 80% combined weight of the biologic cells and the first aqueous solution.

16. The method of claim 5, wherein the shaking in step (b) is conducted for a period of from about 5 seconds to about 10 minutes.

17. The method of claim 16, wherein the shaking is conducted for 2 minutes.

18. The method of claim 5, wherein the DNA released from cells in step (b) is measured by a method selected from the group consisting of: terbium fluorescence, OD₂₆₀ measurement, intercalating dye fluorescence, end point or real time PCR assay, or any combination of the foregoing.

19. The method of claim 5, wherein the separating is conducted by a method selected from the group consisting of: centrifugation, filtration and gravity settling.

20. The method of claim 5, wherein the biologic cells originate from a sample selected from the group consisting of: feces, cell lysate, tissue, blood, tumor, tongue, tooth, buccal swab, phlegm, mucous, wound swab, skin swab, vaginal swab, or any other biological material or biological fluid originally obtained from a human, animal, plant, or environmental sample, including raw samples, complex samples, mixtures, and microbiome samples.

21. The method of claim 5, wherein the biologic cells originate from an organism selected from the group consisting of: spores, biofilms, multicellular organisms, unicellular organisms, prokaryotes, eukaryotes, microbes, bacteria, archaea, protozoa, algae, fungi and viruses.

22. The method of claim 5, wherein the shaking is conducted in the device of claim 1.
